(19) United States
(12) Patent Application Publication
(10) Pub. No.: US 2004/0193276 A1
(43) Pub. Date: Sep. 30, 2004

(54) Modular articulating surface replacement prosthesis

(76) Inventors: Brian J. Maroney, Fort Wayne, IN (US); Jack F. Long, Warsaw, IN (US); Joseph P. Iannotti, Solon, OH (US); Gerald R. Williams JR., Villanova, PA (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/403,577

(22) Filed: Mar. 31, 2003

(51) Int. Cl. A61F 2/40; A61F 2/36; A61F 2/46

(52) U.S. Cl. 623/19.14; 606/102; 623/23.14; 623/23.42

(57) ABSTRACT

A prosthesis (10) for use in performing joint arthroplasty is provided. The prosthesis (10) may be fitted to a humerus (4). The prosthesis (10) includes a body (12) having an articulating surface (14) and an opposed surface (16) at least partially conforming to the humeral head (4). The prosthesis (10) also includes a stem (18) removably attachable to the body (12) for inserting at least partially into the humerus (4).

### CROSS-REFERENCE TO RELATED APPLICATIONS

Cross reference is made to the following applications: DEP 755 entitled “ARTHROPLASTY SIZING GAUGE”, DEP 756 entitled “ARTICULATING SURFACE REPLACEMENT PROSTHESIS”, DEP 5041 entitled “ARTHROPLASTY INSTRUMENT AND ASSOCIATED METHOD”, DEP 5042 entitled “EXTENDED ARTICULATION ORTHOPAEDIC IMPLANT AND ASSOCIATED METHOD” and DEP 5052 entitled “PROSTHETIC IMPLANT, TRIAL AND ASSOCIATED METHOD” filed concurrently herewith which are incorporated herein by reference.

1. Technical Field of the Invention

The present invention relates generally to the field of orthopaedics, and more particularly, to an implant for use in arthroplasty.

### BACKGROUND OF THE INVENTION

The invention relates to implantable articles and methods for implanting such articles. More particularly, the invention relates to a bone prosthesis and a method for implanting the same.

There are known to exist many designs for and methods for implanting implantable articles, such as bone prostheses. Such bone prostheses include components of artificial joints, such as elbows, hips, knees and shoulders. An important consideration in the design and implanting of virtually any implantable bone prosthesis is that the bone have adequate fixation when implanted within the body.

Earlier designs of implantable articles relied upon the use of cement, such as polymethylmethacrylate (PMMA) to anchor the implant. The use of such implants can have some advantages, such as providing a fixation that does not develop free play or does not lead to erosion of joining faces postoperatively. However, the current trend is to use the cements to a lesser extent because of their tendency to lose adhesive properties over time and the possibility that cement contributes to wear debris within a joint.

Recently, implantable bone prostheses have been designed such that they encourage the growth of hard bone tissue around the implant. Such implants are often implanted without cement and the bone grows around surface irregularities, for example, porous structures on the implant.

One such implantable prosthesis is a shoulder prosthesis. During the lifetime of a patient, it may be necessary to replace the natural humeral head and associated glenoid cavity with a prosthesis. Such a shoulder replacement procedure may be necessary to be performed on a patient as a result of, for example, disease or trauma, for example, disease from osteoarthritis or rheumatoid arthritis.

Most shoulder replacement surgeries today involve the implantation of a total shoulder prosthesis. In a total shoulder replacement procedure, a humeral component having a head portion is utilized to replace the natural head portion of the upper arm bone or humerus. The humeral component typically has an elongated intramedullary stem which is utilized to secure the humeral component to the patient's humerus. In such a total shoulder replacement procedure, the natural glenoid surface of the scapula is restructured or otherwise replaced with a glenoid component that provides a bearing surface for the head portion of the humeral component.

With the average age of patients requiring shoulder arthroplasty decreasing, orthopaedic implant manufacturers are developing “bone-sparing” implants for the initial treatment of degenerative arthritis. While bone-sparing implants for the treatment of hip and knee arthroplasty are becoming quite common, bone-sparing shoulder arthroplasty techniques and prostheses are also being developed.

Shoulder surface replacement prostheses are being developed to replace the articulating surface of the proximal humerus with a minimal bone resection and minimal disruption of the metaphysis and the diaphysis. Current designs use a semi-spherical articular dome with a small stem for rotational stability. The under surface of the articular head is also semi-spherical and meets with a spherically machined humeral head.

Typically, however, arthritis of the gleno-humeral joint causes flattening of the humeral head with a large medial osteophyte. The flat humeral head can cause voids in the bone under the prosthesis resulting in limited contact between the prosthesis and the resected bone and may limit the load transfer capability between the prosthesis and the humerus.

Referring now to FIG. 2, a healthy long bone or, in the form of, for example, a humerus 1 is shown. The humerus 1 includes a head 2 on the proximal end of the humerus 1. The head 2 of a healthy humerus has an arcuate outer periphery. The arcuate outer periphery is generally hemispherical and meets with a concave glenoid cavity 3.

Referring now to FIG. 3, a diseased humerus 4 is shown. The diseased humerus 4 includes a head 5. The head 5 is flattened as shown in FIG. 3. The humerus 4 also has developed a large medial osteophyte 7.

Referring now to FIG. 4, prior art prosthesis 8 is shown in position on the head 5 of diseased humerus 4. The head 5 includes a flattened humeral head area or bony defect 9 that leads to a void 6 between the prosthesis 8 and the bony defect 9.

The size and shape of the humerus and the humeral head of patients vary greatly due to the size, bone structure, gender and other factors, which cause the humerus to vary greatly in size and shape. Further, the progression of the bone disease, for example, osteoarthritis, will affect the degree to which the humeral head is flattened. In order that a properly sized and optimum (bone-sparing) prosthesis is selected for a particular humerus with its flattened humeral head, a wide variety of prosthetic sizes and shapes are necessary. This need for a wide variety of products creates great manufacturing cost as well as inventory cost for the orthopedic manufacturer and hospital at which the surgery is performed.

Therefore, there is a need for a prosthesis that provides for the great variety of configurations of bone sparing prosthetic implants with minimum inventory.

### SUMMARY OF THE INVENTION

The present invention provides for a bone sparing implant for the treatment of hip arthroplasty, which includes a body to be fitted to the humeral head and a separable stem to be attached to the body.

The two-piece bone sparing humeral head resurfacing humeral prosthesis of the present invention includes a body for resurfacing the humeral head and a stem attached to the body for securing the body to the humerus. The separate body and stem permits the combination of various stems and varying bodies to be interchanged to form a wide number of combinations with a minimum number of components.

According to one embodiment of the present invention, a prosthesis for use in performing joint arthroplasty is provided. The prosthesis to be fitted to a humeral head of a humerus. The prosthesis includes a body including an articulating surface and an opposed surface at least partially conforming to the humeral head. The prosthesis also includes a stem removably attachable to the body for inserting at least partially into the humerus.

According to another embodiment of the present invention, a prosthesis to be fitted to a head of a long bone is provided. The prosthesis includes a body including a generally hemispherical surface and an opposed surface at least partially conforming to the head. The prosthesis also includes a stem removably attached to said body for inserting at least partially into the humerus.

According to another embodiment of the present invention, a kit for use in performing joint arthroplasty on a bone is provided. The kit includes a prosthetic member having a generally hemispherical articulating surface and a support surface opposed to the articulating surface. The kit also includes a first stem connectable to the prosthetic member and insertable into the long bone. The kit further includes a second stem having at least one dimension different than the first stem. The second stem is connectable to the prosthetic member and insertable into the long bone. The prosthetic member and the first stem or the second stem may be selectively used to form a properly sized prosthesis to perform the joint arthroplasty.

According to a further embodiment of the present invention, a method for providing joint arthroplasty for a long bone is provided. The method includes the steps of making a measurement of the long bone, providing a plurality of prosthetic bodies, providing a stem, selecting one of the plurality of bodies based upon the measurement of the long bone, assembling the stem onto the selected body to form a prosthesis, and implanting the prosthesis onto the long bone.

The technical advantages of the present invention includes the ability to provide better fitting prostheses to match the configuration of the natural humerus and humeral head. For example, according to one aspect of the present invention, a bone sparing humeral prosthesis is provided. The bone sparing prosthesis includes a hollow hemispherical body and a separable stem, with the stem and body being removably connected by a connector. By providing a plurality of bodies and a plurality of stems which each of the respective bodies and stems having a commonly configured connector, the stems and bodies can be mixed and matched to provide for a better fitting prosthesis with a minimal number of components. Thus the present invention provides for a better fitting prostheses.

The technical advantages of the present invention further include the accommodating of multiple sizes and shapes of prostheses. For example, according to one aspect of the present invention, a bone sparing implant can be provided with a hollow hemispherical body for fitting to the humeral head and a stem removably connected to the body. In fact a plurality of bodies and stems may be provided. Thus, the present invention provides for a multitude of sizes and shapes of prostheses.

Another technical advantage of the present invention includes the ability to reduce the cost of bone sparing prosthetic implants. For example, according to one aspect of the present invention, a bone sparing orthopedic implant for attachment to the head of a long bone includes a body for attachment to the head and a stem to secure the body to the head. The body and the head are removably securable by a connection. By providing a common connection to a plurality of bodies and stems and interchanging the bodies and stems, a wide variety of combinations can be provided with a minimal number of different components. By minimizing the number of components, the cost to provide this range of prosthetic sizes and shapes is minimized. Thus, the present invention provides for cost reduction of bone sparing implants.

Another technical advantage of the present invention includes the reduction of inventory. For example, according to one aspect of the present invention, a bone sparing prosthesis for connection to the head of a long bone is provided with a body for attachment to the head of the long bone and a stem for securing the body to the head. By providing a plurality of bodies and stems each having a common connector for removably connecting the stems and the bodies, a wide variety of product offerings can be had with a minimal number of components thereby reducing inventory. Thus the present invention provides for inventory reduction.

Other technical advantages of the present invention will be readily apparent to one skilled in the art from the following figures, descriptions and claims.

### BRIEF DESCRIPTION OF THE DRAWINGS

For a more complete understanding of the present invention and the advantages thereof, reference is now made to the following description taken in connection with the accompanying drawings, in which:

FIG. 1 is a plan view partially in cross section of a modular surface replacement prosthesis according to the present invention including a hemispherical cup and a separate stem with the components being interconnected with a tapered connection;

FIG. 2 is a plan view of a healthy humerus;

FIG. 3 is a plan view of a diseased humerus;

FIG. 4 is a plan view partially in cross section of a prior art humeral prosthesis;

FIG. 5 is an exploded plan view partially in cross section of the modular surface replacement prosthesis of FIG. 1;

FIG. 5A is a plan view of a stem for another embodiment of a modular surface replacement prosthesis having a threaded connection;

FIG. 5B is a plan view of a stem for another embodiment of a modular surface replacement prosthesis having a press-fit connection;

FIG. 6 is a plan view partially in cross section of another embodiment of a surface replacement prosthesis according to the present invention including a spacer and a hemispherical cup having a threaded tapered stem;

FIG. 7 is an exploded plan view partially in cross section of another embodiment of a surface replacement prosthesis according to the present invention including a modular prosthesis including a hemispherical cup and a plug, the prosthesis also including two spacers and a separate stem, with the components being interconnected with a tapered connection;

FIG. 8 is a plan view of the prosthesis of FIG. 7;

FIG. 9 is a plan view partially in cross section of another embodiment of a surface replacement prosthesis according to the present invention including a spacer and a hemispherical cup having a tapered stem;

FIG. 10 is a plan view partially in cross section of another embodiment of a surface replacement prosthesis according to the present invention including an integral prosthesis and stem with a spacer bolted to the prosthesis;

FIG. 11 is a plan view partially in cross section of another embodiment of a surface replacement prosthesis according to the present invention including an integral stem and spacer threaded to the prosthesis;

FIG. 12 is a plan view partially in cross section of another embodiment of a surface replacement prosthesis according to the present invention including an integral cup and stem with a spacer threaded to the cup and having a porous coating on the spacer and the prosthesis;

FIG. 13 is a plan view partially in cross section of a surface replacement prosthesis including a modular prosthesis including a stem and spacer secured to the prosthesis with a tapered connection and with the prosthesis and having a porous coating on the spacer and the stem;

FIG. 14 is a plan view of a kit for use in performing shoulder arthroplasty surgery according to a further embodiment of the present invention;

FIG. 15 is a plan view of kit including gauges for use in performing shoulder arthroplasty surgery according to a further embodiment of the present invention;

FIG. 16 is a plan view partially in cross section of a gauge for determining the appropriate spacer for a surface replacement prosthesis according to the present invention for use on a diseased humerus;

FIG. 17 is a perspective view of a trial for use in performing shoulder arthroplasty surgery with the prosthesis according to a further embodiment of the present invention;

FIG. 18 is a plan view of the trial of FIG. 17;

FIG. 19 is a plan view of a set of instruments including gauges for use in performing shoulder arthroplasty surgery according to a further embodiment of the present invention;

FIG. 20 is plan view of a cutter used to prepare a humerus for implantation of a prosthesis according to the present invention;

FIG. 21 is a flow chart of a method of performing arthroplasty according to the present invention; and

FIG. 22 is a plan view partially in cross section of a modular hip prosthesis for use in performing hip arthroplasty on a femoral head according to another embodiment of the present invention.

### DETAILED DESCRIPTION OF THE INVENTION

Embodiments of the present invention and the advantages thereof are best understood by referring to the following descriptions and drawings, wherein like numerals are used for like and corresponding parts of the drawings.

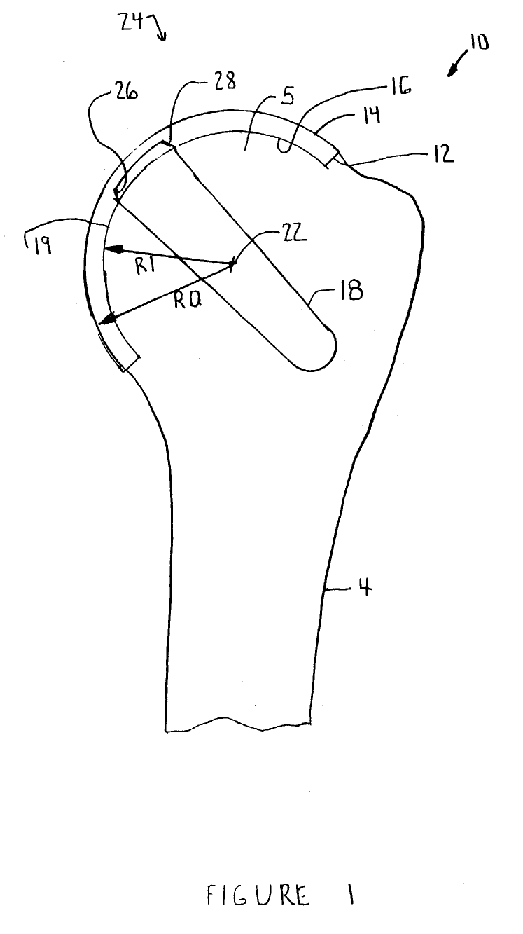

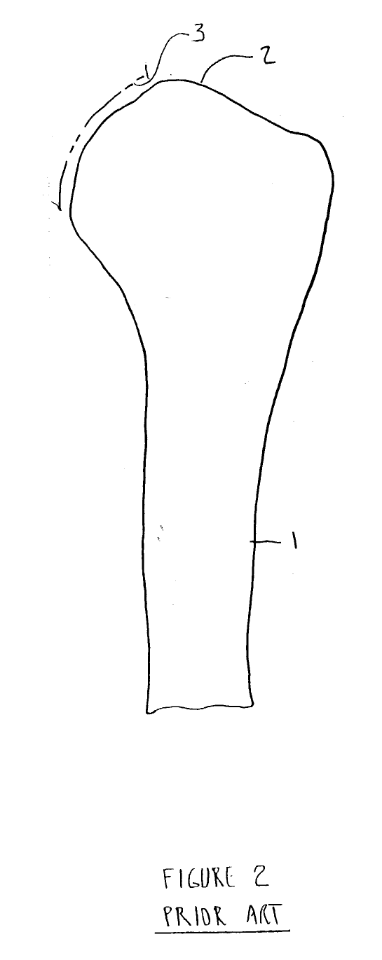

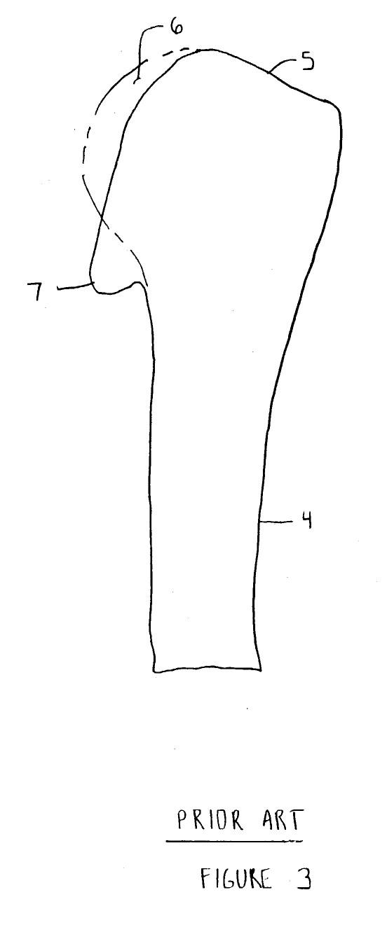

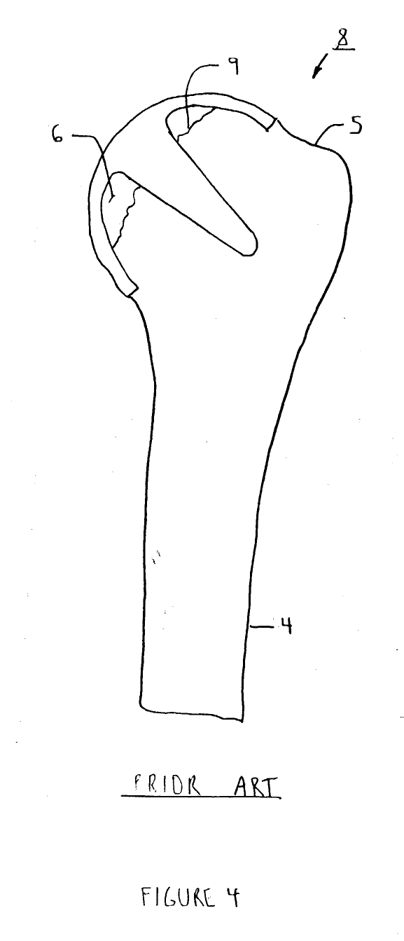

Referring now to FIG. 1, an embodiment of the present invention is shown as prosthesis 10. The prosthesis 10 is used for performing joint arthroplasty. For example, shoulder joint surgery. In particular, the prosthesis 10 may be used for conservative bone-sparing surgery, in which the greater portion of the humeral head is spared. The prosthesis 10 includes a body 12. The body 12 includes an articulating surface 14. The articulating surface 14 cooperates with the glenoid cavity. The glenoid cavity may include a glenoid prosthesis (not shown) or a natural glenoid cavity. The body 12 further includes an opposed surface 16, opposed to the articulating surface 14. The opposed surface 16 at least partially conforms to the humeral head 5.

The prosthesis 10 further includes a stem 18. The stem 18 is removably attachable to the body 12. The stem 18 is utilized for inserting the stem 18 at least partially into the humerus 4.

The body 12 may have any suitable configuration capable of providing a prosthesis for fitting into the humeral head of a humerus. For example, and as shown in FIG. 1, the opposed surface 16 may contact prepared surface 19 of the head 5 of the humerus 4 with the opposed surface 16 serving as a support surface for supporting the body 12 of the prosthesis.

The body 12 of the prosthesis 10 may have any shape and may, as shown in FIG. 1, have a generally hollow hemispherical shape. For example, as shown in FIG. 1, the body 12 may be generally defined by convex articulating surface 14 and the convex opposed surface 16. The opposed surface 16 may be defined by a radius R1 extending from center point 22. The articulating surface 14 may be defined by radius R0 extending from center point 22.

The stem 18 may be removably attached to the body 12 by any suitable method. For example, as shown in FIG. 1, the stem 18 may be connected to the body 12 by means of connector 24. The connector 24 may, as shown in FIG. 1, include an internal taper 26 formed on the body 12, which meets with external taper 28 formed on the periphery of the stem 18.

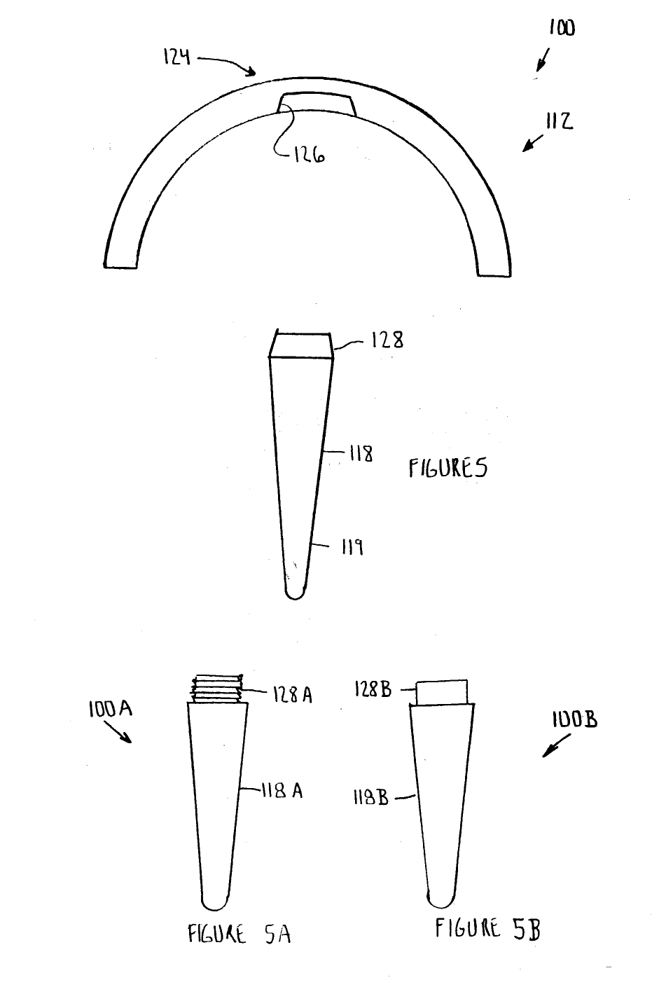

Referring now to FIGS. 5, 5A and 5B, another embodiment of the present invention is shown as prosthesis 100. The prosthesis 100 is similar to the prosthesis 10 of FIG. 1 and includes a hollow hemispherical cup 112, which is removably connected to stem 118. The stem 118 includes a tapered cylindrical body 119. The cup 112 and the stem 118 are joined by, for example, a connector 124 in the form of an external taper 128 on the stem 118, which mates with an internal taper 126 formed in the body 112.

Referring now to FIG. 5A, another embodiment of the present invention is shown as prosthesis 100A. Prosthesis 100A includes a stem 118A which is similar to the stem 118 of FIG. 5 except that stem 118A includes a threadable connector 128A, including external threads to threadably engage with internal threads (not shown) on the body (not shown) of the cup (not shown) of the prosthesis 100A.

Another embodiment of the present invention is shown as prosthesis 100B, as shown in FIG. 5B. The prosthesis 100B includes a tapered stem 118B similar to the stem 118A of FIG. 5A; except that the stem 118B includes a cylindrical end forming a press fit connector 128B. The press fit connector 128B mates with a cylindrical counter bore (not shown) in the body (not shown) of the prosthesis 100B.

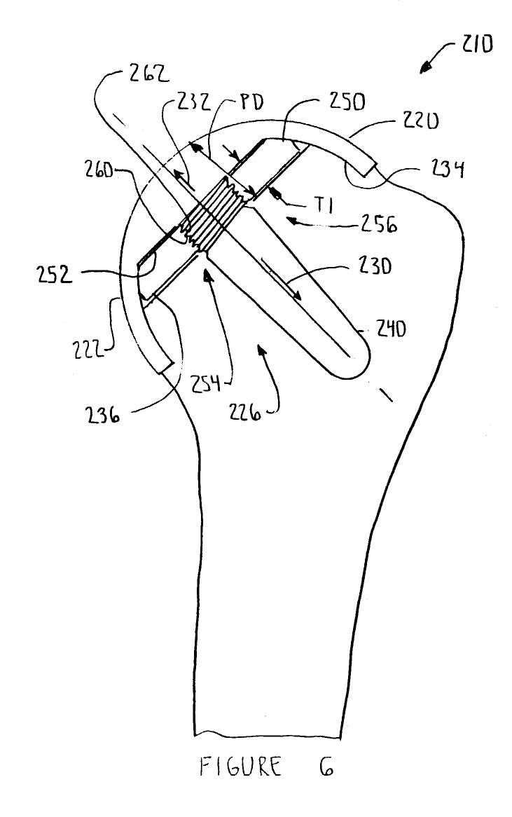

Referring now to FIG. 6, an example of a multi-piece prosthesis is shown as prosthesis 210. Prosthesis 210 of FIG. 5 is similar to the prosthesis 10 of FIG. 1 except that the prosthesis 210 includes a planar support surface unlike the arcuate support surface of the prosthesis 10 of FIG. 1. As shown in FIG. 6, the prosthesis 210 includes in addition to body 220, a spacer 250. The spacer 250 provides for a variety of locations of planar portion 236 of support surface 226.

Thus, by utilizing the prosthesis 210, a common body 220 may be used with a variety of spacers 250 having different thicknesses T1. Thus, for any prosthesis 210 a plurality of planar dimensions PD may be provided by merely changing the spacer 250 to either a thinner or a thicker spacer.

As shown in FIG. 6, the prosthesis 210 includes the body 220. The body 220 is similar to the body 12 of the prosthesis 10 of FIG. 1 in that it includes an articulating surface 222 extending in a second direction 232, as well as, a stem 240 extending in a first direction 230 opposed to the second direction 232. The stem 240 is similar to stem 18 of the prosthesis 10.

As shown in FIG. 6, the body 220 includes a body planar surface 252 to which the spacer 250 is placed. The spacer 250 defines the planar portion 236 of the support surface 226 and works in conjunction with arcuate surface 234 of the body 220 to support the prosthesis 210 against the humerus wall.

As shown in FIG. 6, the spacer 250 preferably has a pair of spaced apart parallel faces defined with the thickness T1. The spacer 250 has a central opening 254 to permit the spacer 250 to be positioned in place against the body planar surface with the stem 240 passing through the opening 254.

Preferably, and as shown in FIG. 6, the spacer 250 is secured to the body 220 by, for example, a connector 256. The connector 256 may, as shown in FIG. 6, be in the form of a threadable connection. For example, the connector 256 may include external threads 260 located on the stem 240. The external threads 260 on the stem 240 cooperate with matching internal threads 262 on the spacer 250. A feature (not shown) in the form of, for example, a recess on the planar portion 236 of the spacer 250 may be utilized to secure the spacer 250 against the body 220.

The body 220 and the spacer 250 may be made of a similar material to that of the body 12 of the prosthesis 10. Thus, for example, the body 220 and the spacer 250 may be made of a cobalt chromium alloy, a titanium alloy or a stainless steel alloy.

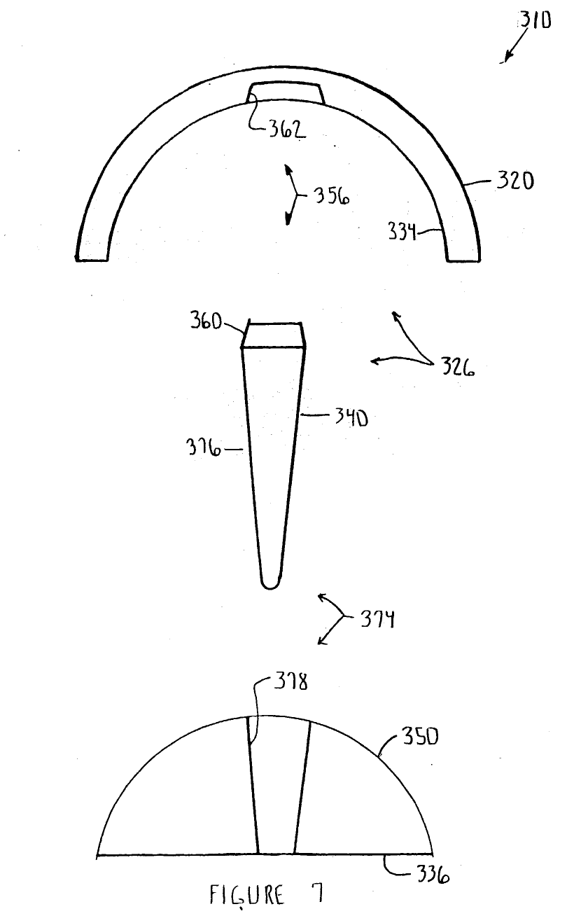

Referring now to FIG. 7, another embodiment of the present invention is shown as prosthesis 310. Prosthesis 310 is similar to prosthesis 10 of FIG. 1 but includes three components, a body 320 similar to body 12 of the prosthesis 10 of FIG. 1 and a stem 340 similar to the stem 18 of the prosthesis 10 of FIG. 1. The prosthesis 310 also includes a plug 350.

While the prosthesis 310 similar to the prosthesis 10 has its components interconnected by means of tapered connections, the prosthesis 310 is different from the prosthesis 10 of FIG. 1. For example, the prosthesis 310 includes a first connector 356 in the form a tapered connection. The tapered connection 356 includes an external taper 360 formed on the stem 340 which connects with an internal taper 362 formed on the body 320. The plug 350 is secured to the stem 340 by means of a second tapered connection 374. The second tapered connection 374 includes an external taper 376 formed on the stem 340 which connects with an internal taper 378 formed on the plug 350. The plug 350 includes a support surface 336 that, together with arcuate surface 334 of the body 320 form support surface 326 of the prosthesis 310 for the securing the prosthesis 310 to the humerus 4.

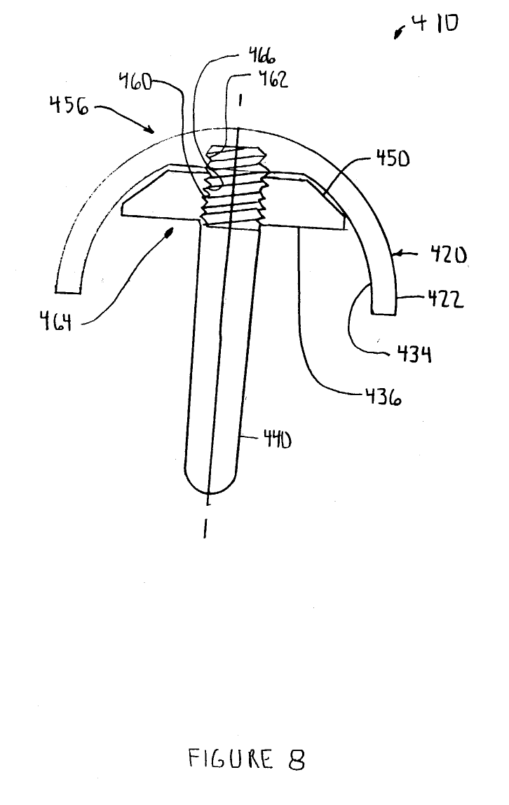

Referring now to FIG. 8, another embodiment of a multi-piece prosthesis is shown as prosthesis 410. The prosthesis 410 is similar to the prosthesis 210 of FIG. 6 and includes a body 420, as well as, a spacer 450. The prosthesis 410 further includes a stem 440. The body 420 of the prosthesis 410 is different than the body 120 of the prosthesis 110 in that the body 420 does not include the stem 440. In the prosthesis 410 of FIG. 8, the stem 440 is a separate component.

As shown in FIG. 8, the spacer 450 is contained between the stem 440 and the prosthesis 410. The body 420 as shown in FIG. 8 has a generally hollow hemispherical shape having a convex outer articulating surface 422 and a convex arcuate support surface 434.

A first connector 456 is used to secure the stem 440 to the body 420. The connector 456 may, as shown in FIG. 8, be in the form internal threads 462 in the body 420 which mate with corresponding external threads 460 on the stem 440. A second connector 464 is used to secure the spacer 450 to the stem 440. The second connector 464 may be in the form internal threads 466 on the spacer 450 which mate with the external threads 460 on the stem 440. It should be appreciated that alternatively the second connector 464 may be in the form of a shoulder extending from the outer periphery of the stem 440 which mates with planar portion 436 of the spacer 450.

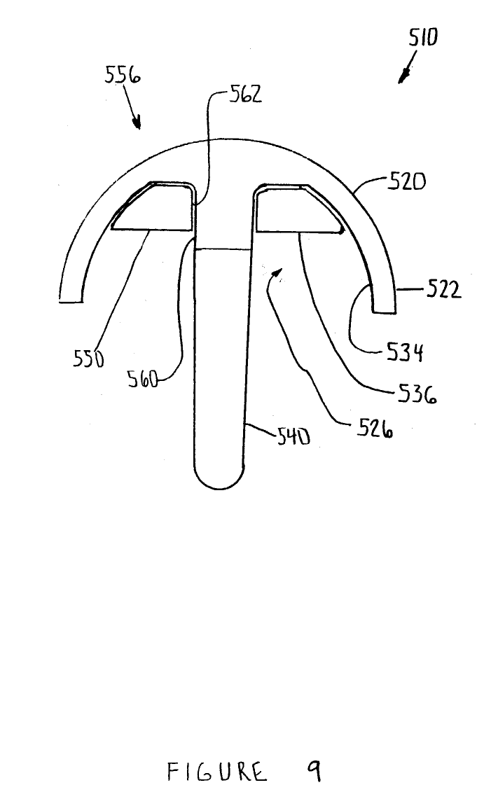

Referring now to FIG. 9, another embodiment of the present invention as prosthesis 510. Prosthesis 510 includes a body 520 similar to the body 220 of the prosthesis 210 of FIG. 6 in that the body 520 includes stem 540 similar to stem 240 of FIG. 6. The prosthesis 510 further includes a spacer 550 similar to the spacer 250 of the prosthesis 210 of FIG. 6.

The spacer 550 is secured to the body 520 by means of a connector 556. The connector 556 is different than the connector 256 of the prosthesis 210 in that the connector 556 is in the form of a taper fit. The spacer 550 includes a tapered opening 562, which engages with tapered stem portion 560 of the stem 540 of the prosthesis 510.

The body 520 includes an articulating surface 522 and an opposed arcuate support surface 534. The spacer 550 includes a planar support surface 536, which together with the arcuate support surface 534 forms support surface 526 for supporting the prosthesis 510 within the humerus 4.

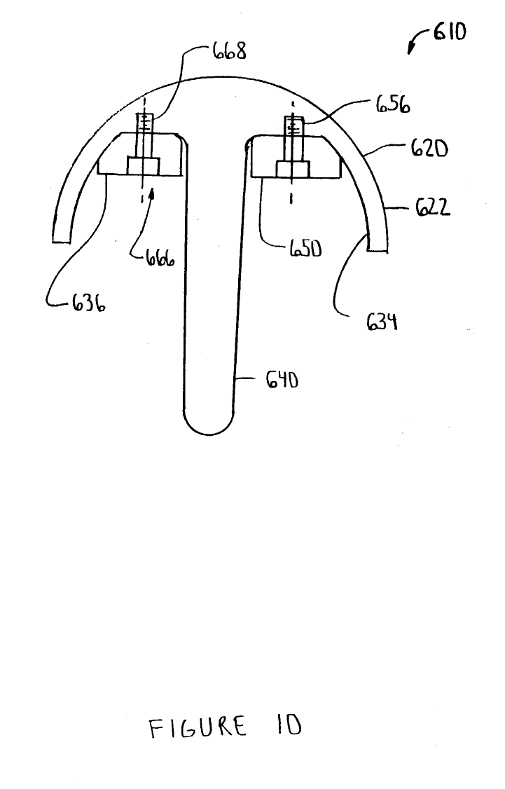

Referring now to FIG. 10, another embodiment of the present invention is shown as prosthesis 610. The prosthesis 610 of FIG. 10 is similar to the prosthesis 510 of FIG. 9, and includes a body 620 similar to the body 520 of FIG. 9. The body 620 includes an articulating surface 622 and an opposed arcuate support surface 634. The body 620 is integral with a stem 640 similar to the stem 540 of FIG. 9. The prosthesis 610 further includes a spacer 650 similar to the spacer 550 of the prosthesis 510 of FIG. 9.

The spacer 650 is secured to the body 620 of the prosthesis 610 by means of a connector 656, which is different than the connector 556 of the prosthesis 510 of FIG. 9. The connector 656 is in the form of a plurality of socket head hex cap screws. The cap screws 656 are fitted through recessed openings 666 in the spacer 650. The cap screws 656 are secured to the body 620 by a plurality of threaded openings 668. The spacer 650 provides planar support surface 636.

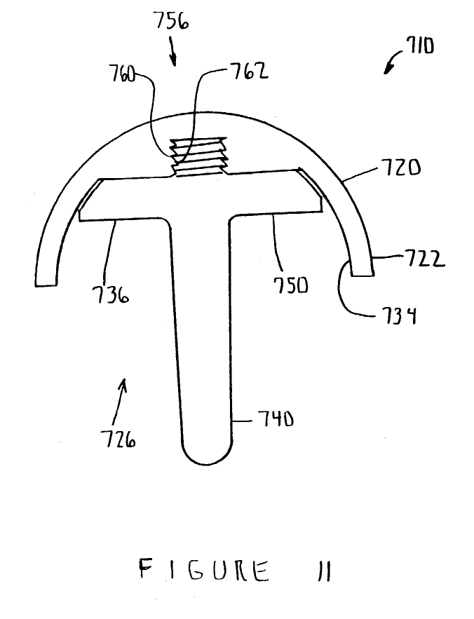

Referring now to FIG. 11, another embodiment of the present invention is shown as prosthesis 710. Prosthesis 710 is similar to the prostheses 210, 410 and 510 in that the prosthesis 710 includes a body 720, a spacer 750, and a stem 740. The prosthesis 710 is different than the prostheses 210, 410 and 510 in that the spacer 750 and the stem 740 are integral with each other. The body 720 of the prosthesis 710 thus does not include the stem 740 and is a separate part from the spacer 750 and the stem 740. As shown in FIG. 11, the body 720 has a generally hollow hemispherical shape having an articulating surface 722 and an opposed arcuate support surface 734. The spacer 750 has a general disc shape with the stem 740 having a generally cylindrical shape and extending outwardly from the center portion of the spacer 750. The spacer 750 is secured to the body 720 by means of a connector 756.

The connector 756, as shown in FIG. 9, is in the form of a threaded stem extending from the spacer 750 in a direction opposed to the stem 740. The connector 756 includes external threads 760, which mate with internal threads 762 in the body 720. The spacer 750 forms planar support surface 736, which together with the arcuate support surface 734 forms support surface 726 for supporting the prosthesis 710 against the humerus 4.

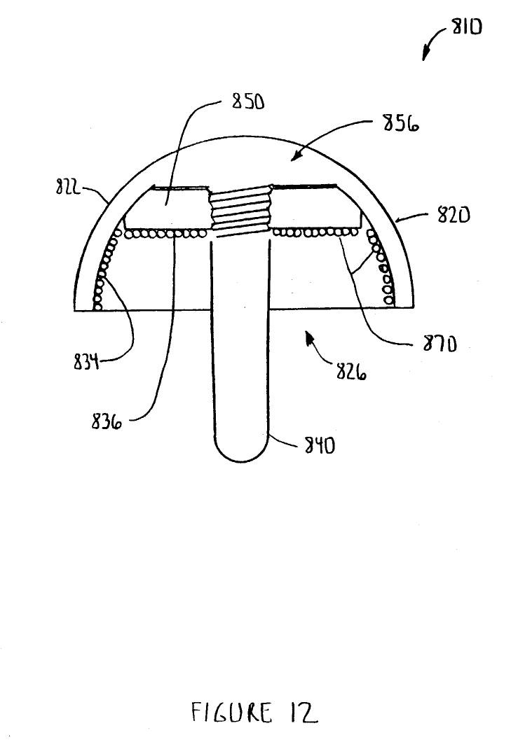

Referring now to FIG. 12, another embodiment of the present invention as shown as prosthesis 810. Prosthesis 810 is similar to the prosthesis 210 of FIG. 6. Prosthesis 810 includes a body 820 similar to the body 220 of FIG. 6 and includes an articulating surface 822 and opposed arcuate support surface 834. The body 820 includes a stem 840 similar to the stem 240 of FIG. 6. The prosthesis 810 further includes a spacer 850 similar to the spacer 250 of FIG. 6. The spacer 850 includes a planar support surface 836, which together with the arcuate support surface 834 serve to form support surface 826 for supporting the prosthesis 810 against the humerus 4. The prosthesis 810 further includes a connector 856 similar to the connector 256 of the prosthesis 210 of FIG. 6.

Unlike the prosthesis 210, the prosthesis 810 includes a porous coating 870 located on the planar support surface 836 and the arcuate support surface 834. The porous coating 870 serves to provide additional surface for promoting bony ingrowth into the prosthesis 810 for improved fixation of the prosthesis 810 to the humerus 4. Any suitable commercially available porous coating may be suitable for the coating 870. For example, the coating may be in the form of POROCOAT®, a product of the assignee of the instant application. More information regarding the coating may be available by referring to U.S. Pat. No. 3,855,638 to Pilliar, incorporated herein by reference in its entirety.

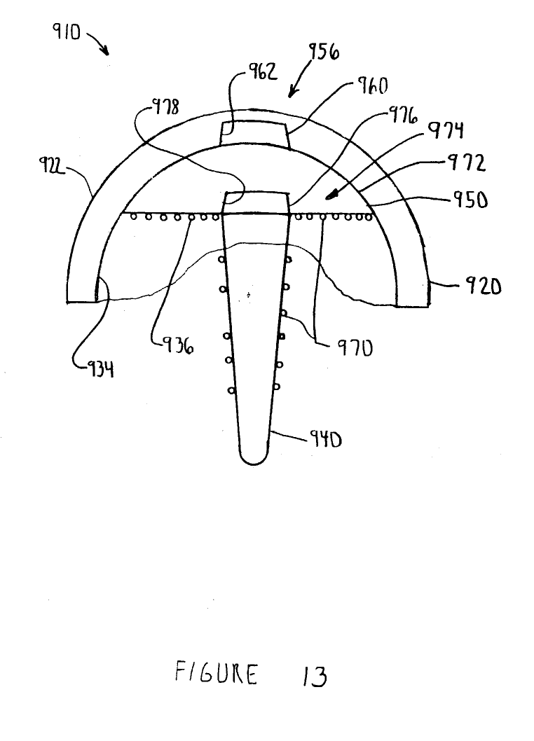

Referring now to FIG. 13, another embodiment of the present invention is shown as prosthesis 910. Prosthesis 910 is a three-part prosthesis including a body 920 similar to the body 12 of the prosthesis 10 of FIG. 1. The body 920 includes a hemispherical outer articulating surface 922 and a concave internal arcuate support surface 934. The prosthesis 910 further includes a plug 950, which serves the purpose of the spacer 250 of the prosthesis 210 of FIG. 6. The plug 950 includes a planar support surface 936 and an opposed spherical outer surface 972 which mates with the arcuate support surface 934 of the body 920. The plug 950 may be secured to the body 920 by any suitable method. For example, as shown in FIG. 11, a first connector 956 in the form of a taper connection is shown.

The first connector 956 includes an exterior taper 960 extending from the plug 950, which mates with an internal taper 962 in the body 920. The prosthesis 910 further includes a generally cylindrical tapered stem 940, which is secured to the plug 950 by a second connector 974.

The stem 940 may be secured to the plug 950 by, for example, the second connector 974. The second connector 974 may have any suitable configuration and may, as shown in FIG. 11, be in the form of an external taper 976 located on the stem 940, which cooperates with an internal taper 978 formed in the plug 950.

As shown in FIG. 13, the prosthesis 910 may further include a coating 970 in the form of, for example, a porous coating, for example, POROCOAT® to encourage ingrowth to assist in the securement of the prosthesis 910 to the humerus 4. The coating 970 may be secured to the stem 940 as well as to the arcuate support surface 934, as well as the planar support surface 936.

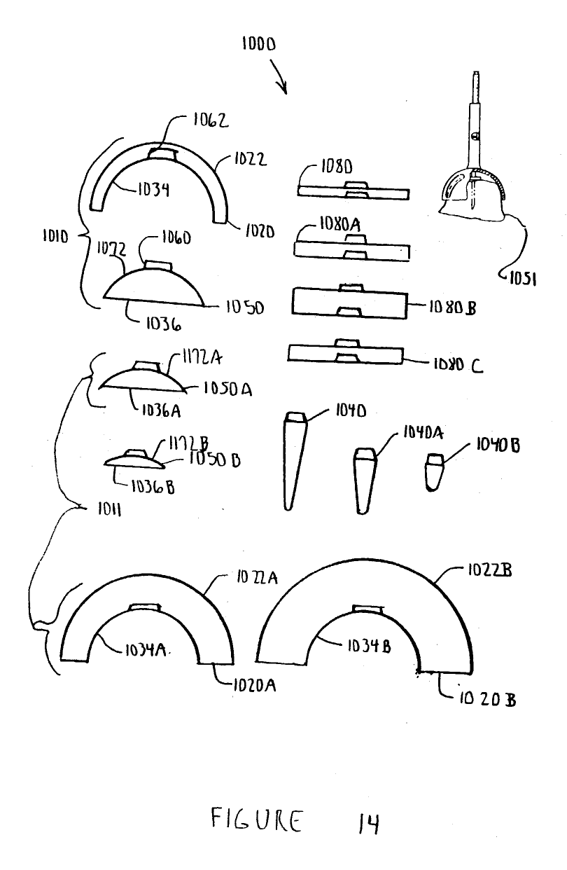

Referring now to FIG. 14, another embodiment of the present invention in the form of kit 1000 is shown. Kit 1000 is similar to kit 900 of FIG. 13 but includes additional components so that patients with greatly varying humeral sizes as well as varying conditions of the flattening of the humeral head may be accommodated within the kit 1000.

For example, as shown in FIG. 14, the kit 1000 includes a plurality of cups, plugs, spacers and stems so that a wide variety of patient humeral conditions can be accommodated. As shown in FIG. 14, the kit 1000 includes a first cup 1020 having a first size articulating surface 1022. The kit 1000 also includes a second cup 1020A. The cup 1020A includes an articulating surface 1022A, which is larger than articulating surface 1022. The kit 1000 may also include a third cup 1020B, having an articulating surface 1022B, which is larger than the articulating surface 1022A of the cup 1020A.

So that the cups 1020, 1020A and 1020B may be utilized with common spacers, plugs and stems, preferably and as shown in FIG. 14, the cup 1020 has an internal arcuate surface 1034 which is the same size and shape as the articulating surface 1034A of the cup 1020A which is also the same size and shape as articulating inner surface 1034B of the cup 1020B.

The kit 1000 further includes a first plug 1050 having a planar surface 1036 and an opposed arcuate surface 1072. The arcuate surface 1072 of the first plug 1050 matingly fits against the arcuate surface 1034 of the first cup 1020. The kit 1000 further includes a second plug 1050A, as well as a third plug 1050B.

The first plug 1050, the second plug 1050A and the third plug 1050B, preferably, each have a respective arcuate periphery 1072, 1072A and 1072B which all matingly fit with the arcuate surface 1034 of the cup 1020. Thus, the first plug 1050, the second plug 1050A and the third plug 1050B may be selectively mated with the first cup 1020. The first plug 1050, the second plug 1050A and the third plug 1050B each have a respective support surface 1036, 1036A and 1036B which provide for varying amounts of resection of the humerus.

The kit 1000 further includes a first spacer 1080, a second spacer 1080A, and a third spacer 1080B and a fourth spacer 1080C. Each of the spacers 1080, 1080A, 1080B and 1080C has a different thickness to accommodate a different amount of resection of the humerus.

The kit 1000 may further include a plurality of stems, for example, a first stem 1040, a second stem 1040A, and a third stem 1040B. Each of the stems 1040, 1040A and 1040B has a different length to accommodate a different size humerus.

Preferably, and as shown in FIG. 14, for the components of the kit 1000 to be able to be easily matched, the components have external tapers 1060 which are all identical as well as internal tapers 1062 which are all identical, so that any internal taper 1062 may fit against an external taper 1060.

For example, as shown in FIG. 14, the cup 1020 may be combined with the plug 1050 to form a first prosthetic member 1010 and the second plug 1050A may be combined with the second cup 1020A to form a second prosthetic member 1011.

The kit 1000 may further include instruments 1051 to be used in conjunction with installing and removing the prosthesis.

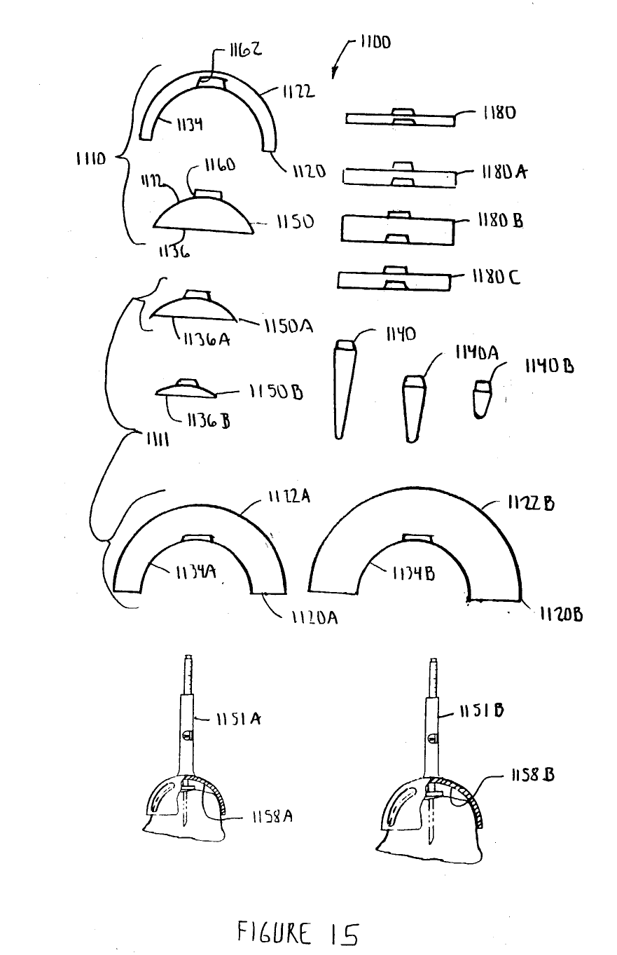

Referring now to FIG. 15, another embodiment of the present invention is shown as kit 1100. The kit 1100 includes a plurality of spacers 1180 similar to the spacers 1080 of the kit 1000 of FIG. 14. The kit 1100 further includes a plurality of cups 1120 similar to the cups 1020 of the kit 1000 of FIG. 14. The kit 1100 further includes a plurality of plugs 1150 similar to the plugs 1050 of the kit 1000 of FIG. 14. The kit 1100 further includes a plurality of stems 1140 similar to the stems 1040 of the kit 1000 of the kit 1000 of FIG. 14.

The kit further includes a first gauge 1151A similar to the gauge 1051 of the kit 1000. The kit 1100 further includes a second gauge 1151B, which is similar to the gauge 1151A. Any one of the cups 1120 may be combined with one of the plugs 1150 to form a prosthesis 1110.

It should be appreciated by utilizing connectors 1162 located on the cups 1120, the plugs 1150, the spacers 1180, and the stems 1140, a prosthetic member may be selected which includes any of the cups 1120, any of the plugs 1150, any of the stems 1140 as well as one or more of the spacers 1180 to form a prosthetic member 1110 according to the present invention.

Continuing to refer to FIG. 15, it should be appreciated that the gauges 1151A and 1151B each have a humeral head contact surface 1158A and 1158B, respectively, which limits the configuration of the prosthesis for which the gauge may be used. Preferably, and as shown in FIG. 15, the gauges 1151A and 1151B are designed such that the respective contact surfaces 1158A and 1158B are sized to correspond to the articulating surface of a particular sized prosthesis.

For example, as shown in FIG. 15, gauge 1151A has a humeral head contact surface 1158A, which has the same size profile as the articulating surface 1122A of the cup 1120A. Similarly, the second gauge 1151B has a humeral head contact surface 1158B, which has the same dimensions as the articulating surface 1122B of the second cup 1120. The kit 1100, therefore, accommodates prostheses having different articulating surfaces.

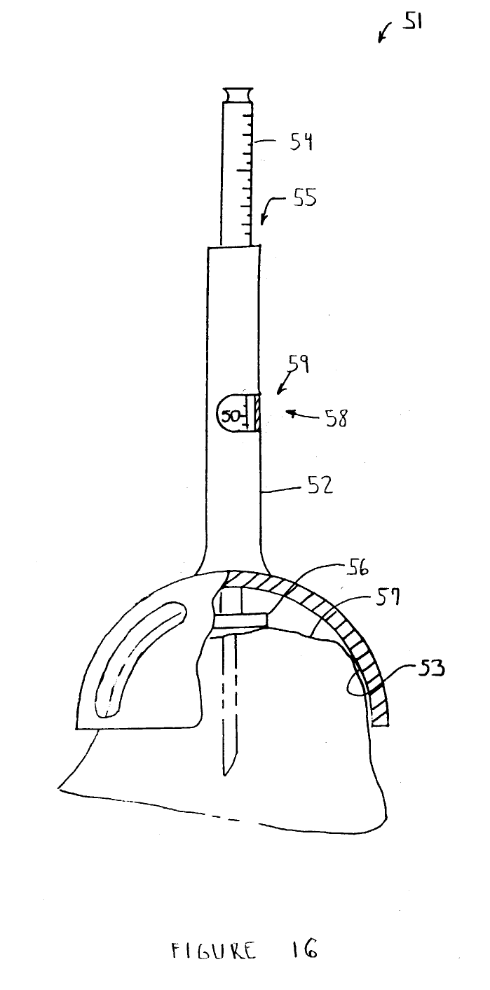

Referring now to FIG. 16, a gauge 51 is shown for use in determining the amount of resection required to the flattened humeral head and the corresponding spacer or spacers required for use with the prosthesis of the present invention. The gauge 51 includes a gauge body 52 including an arcuate contact surface 53, which has a shape similar to that of the interior of the prosthesis to be implanted. A rod 54 is slidably fitted within a longitudinal opening 55 in the gauge body 52. A contact probe 56 is positioned on an end of the rod 54. The contact probe 56 contacts flattened humeral head 57. The position of the contact probe 56 when in contact with the humeral head 57 is measured at a window 58 in the gauge body 52 and indicia 59 on the rod 54 indicate the appropriate amount of resection of the flattened humeral head 57 and the corresponding spacer required because of the resection.

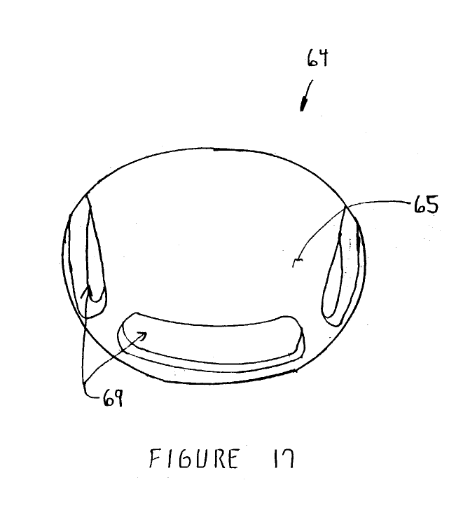

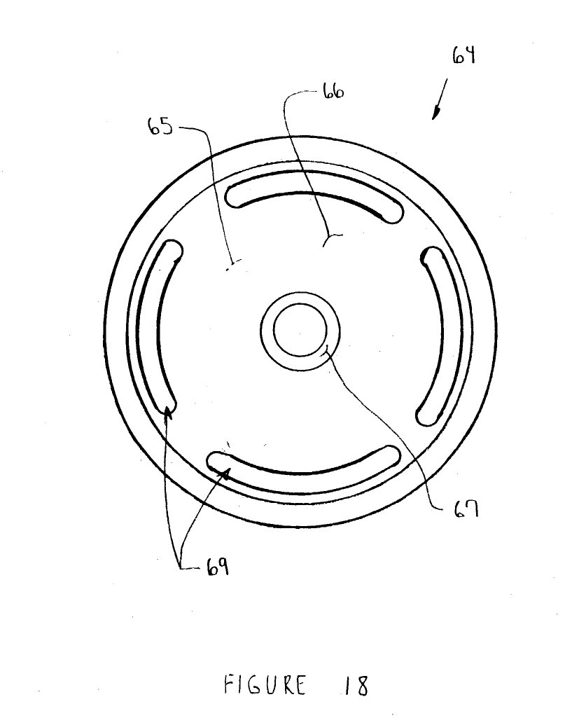

Referring now FIGS. 17 and 18, a trial 64 for use with the prosthesis of the present invention is shown. The trial 64 is utilized during shoulder arthroplasty to verify the proper selection of the prosthetic member by implanting the trial 64 into the humeral head and performing trial reductions on the arm to verify the selection of the particularly sized trial and corresponding prosthesis. The trial 64 is removed and replaced with the corresponding prosthesis. The trial 64 may be reused after sterilization. The trial is made of any suitable durable material and may, for example, be made of a durable plastic that may be sterilized by standard methods such as used in an autoclave.

The trial 64 mimics the size and shape of the prosthesis. The trial 64 therefore includes an articulating surface 65 and an opposed support surface 66. The trial 64 further includes a stem 67 extending outwarding from the support surface 66.

As shown in FIGS. 17 and 18, the trial 64 may also include a plurality of spaced apart openings 69 to assist in the removal of the trial 64.

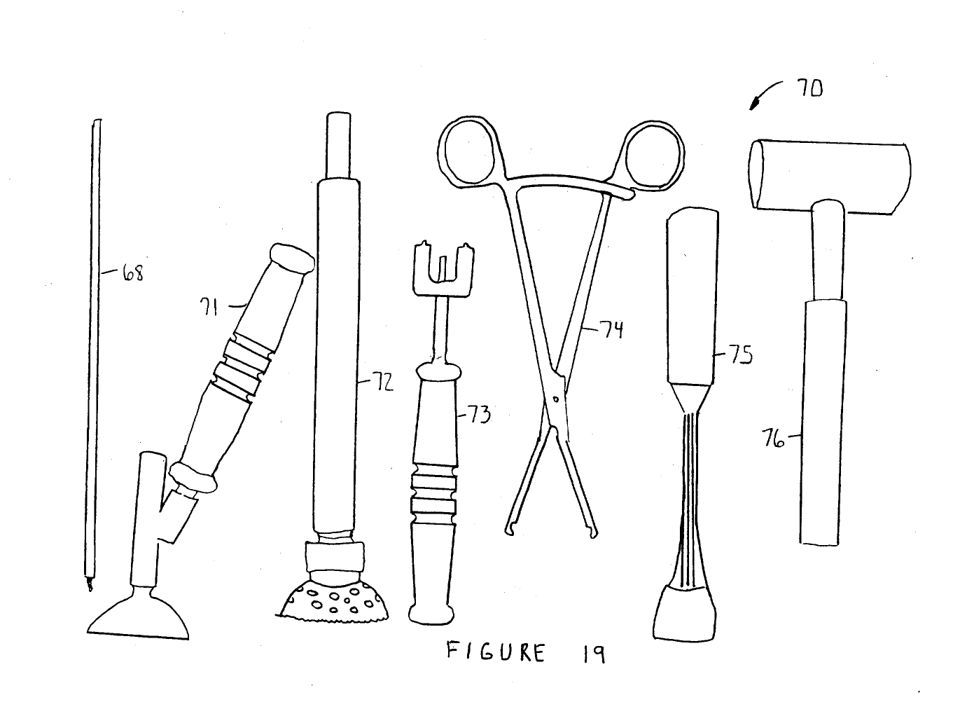

Referring now to FIG. 19, a kit 70 for use when performing an arthroplasty to implant the prosthesis of the present invention. The kit 70 includes guide pin 68, a guide pin alignment tool 71 for assisting in aligning the guide pin and positioning it into the humerus. The instrument kit 70 also includes a cutting tool assembly 72 for preparing the humeral head. The instrument kit 70 further includes a cutting tool assembly wrench 73 for assembling and disassembling the cutting tool from the cutting tool assembly 72. The instrument kit 70 also includes forceps 74 for securely gripping items. The instrument kit 70 also includes a humeral head impactor 75 that is used with surgical mallet 76 to drive the implant into its final set.

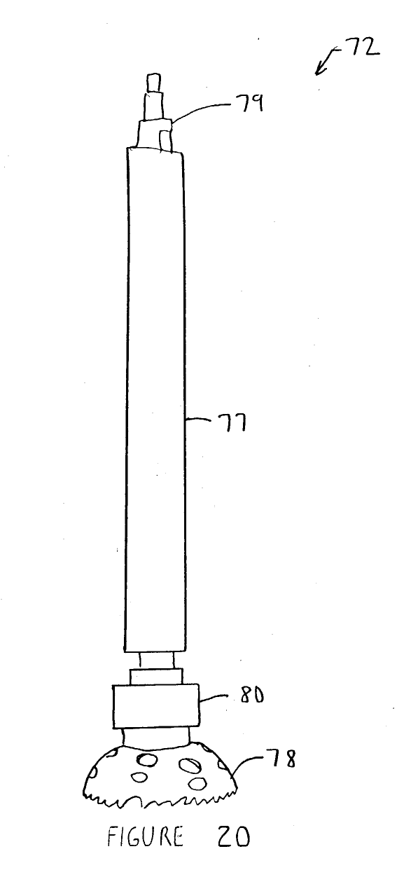

Referring now to FIG. 20, the cutting tool assembly 72 is shown in greater detail. The cutting tool assembly 72 includes a tool holder 77 to which a cutting tool 78 in the form of, for example, a hemispherically shaped reamer is attached. The tool holder 77 includes a drive adapter 79 for attaching a power device (not shown) to the cutting tool assembly 72. The tool holder 77 further includes an adapter 80 for securing the cutting tool 78 to the tool holder 77.

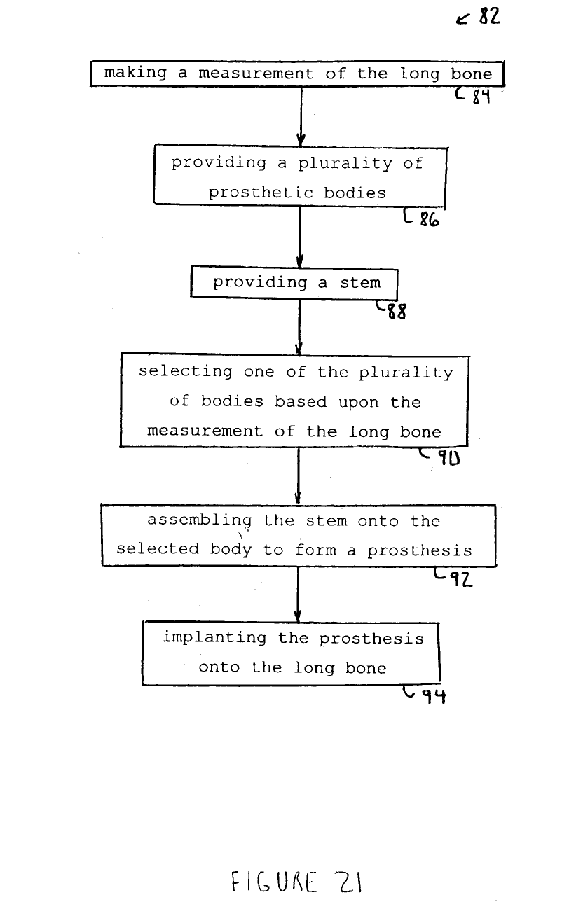

Referring now to FIG. 21, a method for performing a joint arthroplasty is shown as method 82. The method 82 includes a first step 84 of making a measurement of the long bone. The method 82 also includes a second step 86 of providing a plurality of prosthetic bodies as well as a third step 88 of providing a stem. The method 82 further includes a fourth step 90 of selecting one of the plurality of bodies based upon the measurement of the long bone. The method 82 also includes a fifth step 92 of assembling the stem onto the selected body to form a prosthesis. The method 82 further includes a sixth step 94 of implanting the prosthesis onto the long bone.

The method 82 may further include a step (not shown) of providing a gauge for making a measurement of the contour of the long bone. If the providing a gauge step is included, the making of measurement step 84 of the method 82 may include making a measurement of the long bone with the gauge.

The method 82 further includes the step (not shown) of providing a second prosthetic member having at least one dimension different from the first mentioned prosthetic member gauge. The method may also include the step of selecting one of the plurality of prosthetic members based upon the measurement of the long bone.

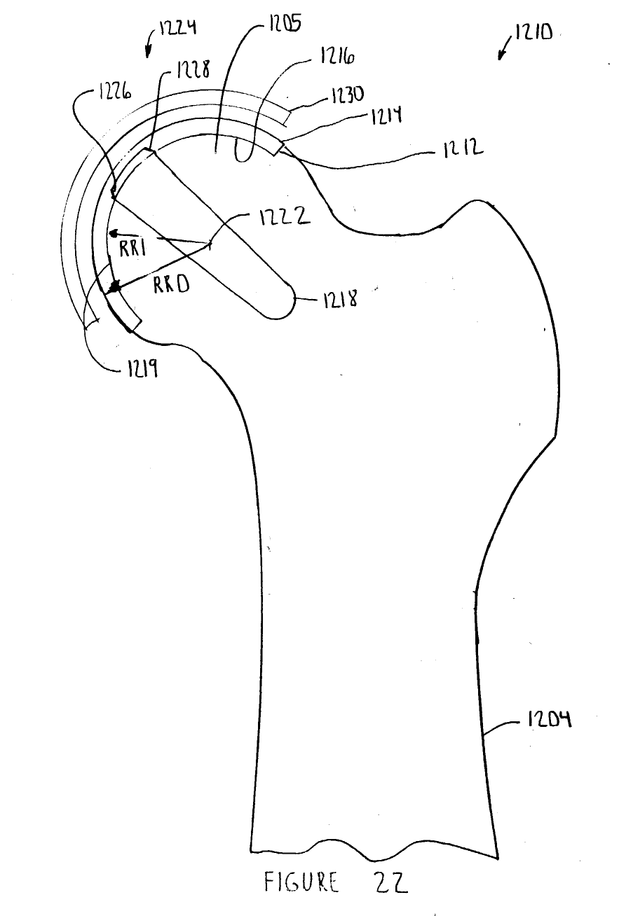

Referring now to FIG. 22, an embodiment of the present invention is shown as prosthesis 1210. The prosthesis 1210 is used for performing hip arthroplasty. In particular, the prosthesis 1210 may be used for conservative bone-sparing surgery, in which the greater portion of the femural head is spared.

The prosthesis 1210 is similar to the prosthesis 10 of FIG. 1, except the prosthesis 1210 is designed for use in a head 1205 of a femur 1204. The prosthesis 1210 includes a body 1212. The body 1212 includes an articulating surface 1214. The articulating surface 1214 cooperates with the acetabulum. The acetabulum may include a hip cup or shell 1230 or a natural acetabulum. The body 1212 further includes an opposed surface 1216, opposed to the articulating surface 1214. The opposed surface 1216 at least partially conforms to the femoral head 1205.

The prosthesis 1210 further includes a stem 1218. The stem 1218 is removably attachable to the body 1212. The stem 1218 is utilized for inserting the stem 1218 at least partially into the femur 1204.

The body 1212 may have any suitable configuration capable of providing a prosthesis for fitting into the femoral head of a femur. For example, and as shown in FIG. 22, the opposed surface 1216 may contact prepared surface 1219 of the head 1205 of the femur 1204 with the opposed surface 1216 serving as a support surface for supporting the body 1212 of the prosthesis.

The body 1212 of the prosthesis 1210 may have any shape and may, as shown in FIG. 22, have a generally hollow hemispherical shape. For example, as shown in FIG. 22, the body 1212 may be generally defined by convex articulating surface 1214 and the concave opposed surface 1216. The opposed surface 1216 may be defined by a radius RR1 extending from center point 1222. The articulating surface 1214 may be defined by radius RR0 extending from center point 1222. The stem 1218 may be removably attached to the body 1212 by any suitable method.

For example, as shown in FIG. 22, the stem 1218 may be connected to the body 1212 by means of connector 1224. The connector 1224 may, as shown in FIG. 22, include an internal taper 1228 formed on the body 1212, which mates with external taper 1226 formed on the periphery of the stem 1218.

Although the present invention and its advantages have been described in detail, it should be understood that various changes, substitutions, and alterations can be made therein without departing from the spirit and scope of the present invention as defined by the appended claims.

We claim:

1. A prosthesis for use in performing joint arthroplasty, said prosthesis to be fitted to a humeral head of a humerus, said prosthesis comprising:
a body including an articulating surface and an opposed surface at least partially conforming to the humeral head; and
a stem removably attachable to said body for inserting at least partially into the humerus.

2. The prosthesis of claim 1, wherein at least a portion of the opposed surface comprises a support surface for supporting the prosthesis against the humerus.

3. The prosthesis of claim 1, further comprising a spacer operably associated with said body and positioned opposed to the articulating surface, said spacer including a support surface for supporting the prosthesis against the humerus.

4. The prosthesis of claim 3, wherein at least one of said body and said spacer comprise a connection for connecting said spacer to said body.

5. The prosthesis of claim 4, wherein said connection comprises at least one of a threaded connection, a press-fit connection, a tapered connection, and a threaded fastener.

6. The prosthesis of claim 1, wherein at least one of said stem and said body includes a portion thereof having a coating to encourage bone ingrowth.

7. The prosthesis of claim 1, wherein said body includes a hemispherical cup.

8. The prosthesis of claim 7, wherein said body includes a plug having a portion conforming to the inner periphery of the cup, said plug including a support surface for supporting the prosthesis against the humerus.

9. A joint prosthesis for use in performing joint arthroplasty, said prosthesis to be fitted to a head of a long bone, said prosthesis comprising:
a body including a generally hemispherical surface and an opposed surface at least partially conforming to the head; and
a stem removably attached to said body for inserting at least partially into the long bone.

10. The joint prosthesis of claim 9, wherein the long bone is a femur.

11. A kit for use in performing joint arthroplasty on a long bone, said kit comprising:
a prosthetic member including a generally hemispherical articulating surface and a support surface opposed to the articulating surface;
a first stem connectable to the prosthetic member and insertable into the long bone; and
a second stem having at least one dimension different than said first stem, said second stem connectable to the prosthetic member and insertable into the long bone, whereby said prosthetic member and one of said first stem and said second stem may be selectively used to form a properly sized prosthesis to perform the joint arthroplasty.

12. The kit of claim 11, further comprising a second prosthetic member including a second prosthetic member articulating surface and having at least one dimension different than said first mentioned prosthetic member.

13. The kit of claim 11, further comprising:
a gauge, said gauge including a gauge body having a gauge contact portion thereof for contact with the bone, the gauge contact portion being shaped to correspond to the contact surface of said prosthetic member.

14. The kit of claim 11, further comprising:
a second prosthetic member including a second prosthetic member articulating surface for contact with the bone and having at least one dimension different than said first mentioned prosthetic member;
a first gauge, said first gauge including a first gauge body having a first gauge contact portion thereof for contact with the bone, the first gauge contact portion being shaped to correspond to said first mentioned prosthetic member; and
a second gauge, said second gauge including a second gauge body having a second gauge contact portion thereof for contact with the bone, the second gauge contact portion being shaped to correspond to said second prosthetic member.

15. The kit of claim 11, wherein at least one of said prosthetic member, said first stem and said second stem comprises a connector for connecting said one of said first stem and second stem to said prosthetic member.

16. The kit of claim 11, wherein said connector comprises at least one of a threaded connection, a press-fit connection, a tapered connection, and a threaded fastener.

17. The kit of claim 11, wherein the long bone is one of a humerus and a femur.

18. A method for providing joint arthroplasty for a long bone, comprising:
making a measurement of the long bone;
providing a plurality of prosthetic bodies;
providing a stem;
selecting one of the plurality of bodies based upon the measurement of the long bone;
assembling the stem onto the selected body to form a prosthesis; and
implanting the prosthesis onto the long bone.

19. The method of claim 18:
further comprising the step of providing a gauge for making a measurement of the contour of a long bone; and
wherein the making a measurement step comprises making a measurement of the long bone with the gauge.

20. The method of claim 19, further comprising the steps of:
providing a second prosthetic member having at least one dimension different from the first mentioned prosthetic member gauge; and
selecting one of the plurality of prosthetic members based upon the measurement of the long bone.
